(19) United States
(12) Patent Application Publication
(10) Pub. No.: US 2021/0106969 A1
(43) Pub. Date: Apr. 15, 2021

(54) DEMETHYLATION APPARATUS AND CANCER THERAPY APPARATUS COMPRISING SAME, AND DEMETHYLATION METHOD AND METHOD AND APPARATUS FOR CANCER THERAPY USING SAME

(71) Applicant: UNIVERSITY OF SEOUL INDUSTRY COOPERATION FOUNDATION, Seoul (KR)

(72) Inventors: Joo-Hiuk SON, Seoul (KR); Hwayeong CHEON, Seoul (KR)

(73) Assignee: UNIVERSITY OF SEOUL INDUSTRY COOPERATION FOUNDATION, Seoul (KR)

(21) Appl. No.: 17/044,784

(22) PCT Filed: Nov. 23, 2018
(86) PCT No.: PCT/KR2018/014489

§ 371 Date: Oct. 1, 2020

(30) Foreign Application Priority Data
Apr. 6, 2018 (KR) 10-2018-0040634

(51) Int. Cl. B01J 19/12; A61N 5/06

(52) U.S. Cl. CPC B01J 19/121; A61N 2005/067; A61N 5/062

(57) ABSTRACT

The disclosure relates to a demethylation apparatus and a demethylation method wherein a terahertz wave of a first frequency band including a methylation resonance frequency is irradiated onto a methylated molecule to demethylate the methylated molecule, and to a cancer therapy apparatus including the demethylation apparatus and a cancer therapy method including the demethylation method.

### BACKGROUND OF THE INVENTION

### Technical Field

The disclosure relates to a demethylation apparatus, a cancer therapy apparatus including the same, and a demethylation method and a cancer therapy apparatus and method using the same.

### Background Art

The terahertz electromagnetic wave (also referred to as terahertz wave hereinafter) has a wavelength between those of infrared ray and microwave, that is, a frequency ranging from 0.1 to 10 THz. Since the energy of terahertz wave is lower than the ionization level of biological sample, terahertz wave is used in biological system test. As a result, damage to biological samples caused by biological system test can be minimized.

The function of DNA may be controlled by a chemical change called methylation. When the excessive methylation phenomenon, in which the methylation level deviated from normal level, occurs, the function of DNA becomes abnormal and various diseases may occur.

### DISCLOSURE

### Technical Solution

It is an object of the invention to provide an apparatus and method capable of demethylating methylated molecules, and a cancer therapy apparatus and cancer therapy method using the same.

According to one aspect of the present invention, there is provided a demethylation method, comprising: (a) irradiating a terahertz wave of a first frequency band including a methylation resonance frequency onto a methylated molecule; and (b) demethylating the methylated molecule by the terahertz wave.

The demethylation method further comprises: (c) generating the terahertz wave using a laser beam; and (d) condensing and steering the terahertz wave by a polarizer toward the methylated molecule to be irradiated in (a).

The demethylation method further comprises: (e) filtering the terahertz wave condensed and steered in (d) to pass the terahertz wave of the first frequency band.

The first frequency band comprises a frequency of 1.6 THz.

According to another aspect of the present invention, there is provided a cancer therapy method comprising a demethylation method.

According to yet another aspect of the present invention, there is provided a cancer therapy apparatus capable of performing the demethylation method.

According to yet another aspect of the present invention, there is provided a demethylation apparatus irradiating a terahertz wave of a first frequency band including a methylation resonance frequency onto a methylated molecule to demethylate the methylated molecule by the terahertz wave.

The demethylation apparatus comprises: a laser beam generator generating a laser beam; a crystal excited by the laser beam to generate the a terahertz wave; a polarizer condensing and steering the terahertz wave generated by the crystal toward the methylated molecule to be irradiated onto the methylated molecule.

The demethylation apparatus further comprises a filter passing the terahertz wave of the first frequency band.

The filter is positioned in an optical path between the polarizer and the methylated molecule

The demethylation apparatus further comprises: a first half wavelength plate changing a polarization direction of the laser beam generated by the laser beam generator by 90 degrees; and a grating 300 tilting the laser beam having the polarization direction thereof changed by 90 degrees toward the crystal to be incident thereon.

The demethylation apparatus further comprises: an optical condenser disposed in an optical path between the grating and the crystal and condensing the laser beam from the grating to be incident on the crystal.

The demethylation apparatus further comprises: a second half wavelength plate disposed in an optical path between the optical condenser and the crystal and changing a polarization direction of the laser beam condensed by the optical condenser to a vertical polarization.

The terahertz wave comprises terahertz wave pulses or a continuous terahertz wave.

The first frequency band comprises a frequency of 1.6 THz.

According to yet another aspect of the present invention, there is provided a cancer therapy apparatus comprising the demethylation apparatus.

### Advantageous Effects

A demethylation apparatus capable of demethylating methylated molecules, a cancer therapy apparatus including the same, and a demethylation method and a cancer therapy method using the same are provided through the embodiments.

### DESCRIPTION OF DRAWINGS

FIG. 1 illustrates a demethylation apparatus according to an embodiment of the present invention.

FIG. 2 illustrates a demethylation apparatus according to another embodiment of the present invention.

FIG. 3 illustrates demethylation apparatus including a terahertz wave measuring device according to yet another embodiment of the present invention.

FIG. 4 is a graph showing the demethylation degree of DNA demethylated by high-power terahertz wave using the demethylation apparatus shown in FIG. 2.

FIG. 5 illustrates an experiment for demonstrating the effect of demethylation by manufacturing three samples.

FIG. 6 illustrates a method of manufacturing an M-293T.

FIG. 7 illustrates a method of measuring demethylation results.

FIG. 8 illustrates a sample holder.

FIG. 9 is a graph showing the methylation degrees of the three samples verified by ELISA-like reaction.

FIG. 10 is a graph showing the demethylation degree according to exposure time to high-power terahertz wave.

FIG. 11 is a graph showing the demethylation degrees of three samples exposed to a high-power terahertz wave generated by the demethylation apparatus shown in FIG. 1.

FIG. 12 is a graph showing the demethylation degrees of three samples exposed to a high-power terahertz wave generated by the demethylation apparatus shown in FIG. 2.

FIG. 13 schematically illustrates a process of demethylation experiment.

FIGS. 14 through 18 illustrate graphs depicting resonance frequency of a blood cancer sample and resonance frequency of a demethylated blood cancer sample, and bar graphs depicting methylation degrees of the samples measured by a terahertz wave measuring apparatus and verified by Elisa-like reaction method.

FIG. 19 is a graph showing that demethylation actually occurs in leukemia cell line when irradiated with high-power terahertz wave.

FIG. 20 is a graph showing that demethylation actually occurs in skin cancer cell line and breast cancer cell line when irradiated with high-power terahertz wave.

FIG. 21 illustrates a cancer therapy apparatus according to an embodiment of the present invention.

### MODE FOR INVENTION

Hereinafter, embodiments of the present invention will be described in detail with reference to the accompanying drawings such that those skilled in the art can easily implement the present invention. However, the present invention is not limited to the embodiments described herein and may be embodied in other forms.

In order to clearly describe the present invention, parts unrelated to the description have been omitted, and like reference numerals indicates like parts throughout the specification. However, the present invention is not necessarily limited to the drawings.

The demethylation apparatus using a terahertz wave according to an embodiment irradiates a high-power terahertz electromagnetic wave onto methylated molecules, for example, methylated DNA for demethylation. Hereinafter, the high-power terahertz electromagnetic wave is referred to as a high-power terahertz wave. In the present disclosure, a methylation resonance frequency in a terahertz band is detected, and a demethylation reaction is induced by the high-power terahertz wave of a band including the corresponding resonance frequency.

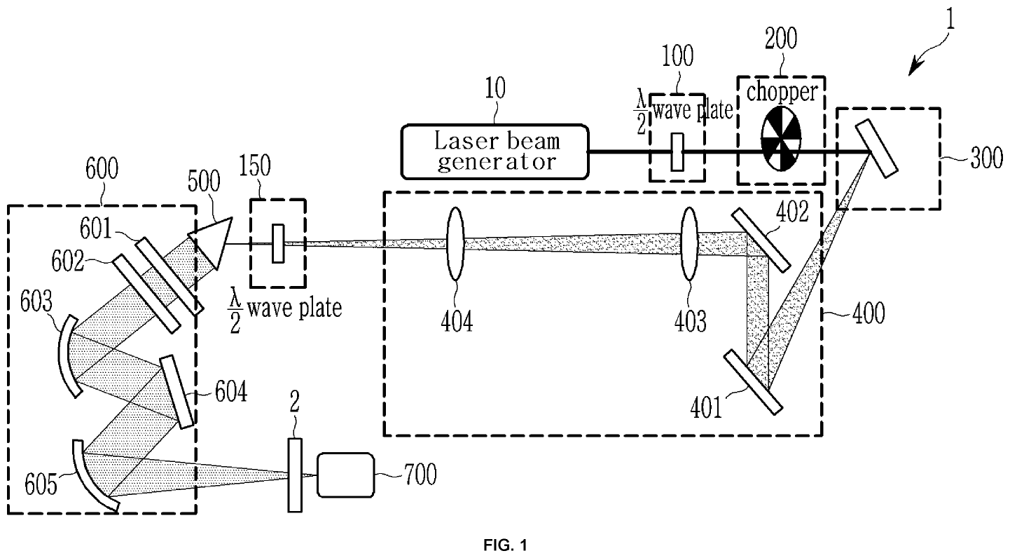

First, a demethylation apparatus using a terahertz wave will be described with reference to FIG. 1.

FIG. 1 illustrates a demethylation apparatus according to an embodiment of the present invention.

A high-power laser generated by a regenerative amplifier is irradiated onto a LiNbO₃ crystal to generate a high-power terahertz wave in the demethylation apparatus 1. A power of the high-power terahertz wave may be measured by a pyroelectric detector in the demethylation apparatus 1. The demethylation apparatus 1 may induce a demethylation reaction by irradiating the high-power terahertz wave onto a methylated molecule (referred to as a sample hereinafter). In particular, since a terahertz wave of a certain band may be advantageous in inducing the demethylation reaction, a terahertz electromagnetic wave of the corresponding band may be generated by using a filter according to the embodiment.

As shown in FIG. 1, the demethylation apparatus 1 includes a laser beam generator 10, a half wavelength plates 100 and 150, an optical chopper 200, a grating 300, a optical condenser 400, a crystal 500, a polarizer 600 and a pyroelectric detector 700.

The laser beam generator 10 generates a high-power laser beam using a titanium:sapphire regenerative amplifier.

The half wavelength plate 100 changes the polarization direction of the high-power laser beam generated by the laser beam generator 10 by 90 degrees such that the laser beam has horizontal polarization before the laser beam is incident on the grating 300. The half wavelength plate 150 changes the polarization direction of the laser beam incident on the crystal 500 by 90 degrees such that the laser beam has a vertical polarization.

The optical chopper 200 intermittently passes i.e. intermittently blocks the laser beam passed through the half wavelength plate 100 at a predetermined period. As a result, the presence and the absence of the laser beam alternately occurs such that a lock-in amplifier (not shown) may generate a detection signal which has the same phase as a reference signal by rectifying the laser beam which has passed through the optical chopper 200.

According to one embodiment, the demethylation apparatus 1 may generate a terahertz wave pulse or a continuous terahertz wave. According to one embodiment, the demethylation method may relate to a method capable of showing that demethylation may be induced through a detection device using an arbitrary terahertz wave generator. The laser beam is pulsed using the optical chopper 200 to generate a high-power terahertz wave pulse. However, the present invention is not limited thereto, and the demethylation apparatus 1 may generate a continuous terahertz wave. In such case, the energy of a continuous terahertz wave may have a level at which demethylation of a sample is induced.

The grating 300 tilts to a specific angle the beam having horizontal polarization passed through the half wavelength plate 100. In order to generate the high-power terahertz wave using the crystal 500, the laser beam should be incident on the crystal 500 at a specific angle. For example, the grating 300 may have a grating angle and a grating spacing such that the laser beam is incident at an angle of 65 degrees with respect to the surface of the crystal 500.

The optical condenser 400 is located in an optical path between the grating 300 and the crystal 500 and narrows the laser beam from the grating 300. Since the laser beam passing through the grating 300 is too wide to be incident on the crystal 500, the optical condenser 400 uses a lens and a mirror to condense the beam to have a cross-section suitable to be incident on the crystal 500.

As shown in FIG. 1, the optical condenser 400 includes two reflectors 401 and 402 and two lenses 403 and 404. The laser beam emitted from the grating 300 is reflected by the two reflectors 401 and 402 and passes through the lens 403, and the beam condensed by the lens 403 and then further condensed by passing through the lens 404. The present invention is not limited to the optical condenser 400 shown in FIG. 1. For example, an appropriate number of lenses may be further used to condense the laser beam to a degree suitable for the crystal 500.

The half wavelength plate 150 is located in the optical path between the optical condenser 400 and the crystal 500, and changes the polarization direction of the laser beam condensed by the optical condenser 400. As a result, the polarization direction of the laser beam incident on the crystal 500 passed through the half wavelength plate 150 is vertical.

The crystal 500 is excited by the incident laser beam and emits a terahertz wave. In the embodiment, crystal is used as an example of the terahertz wave generator, but the present invention is not limited thereto. The demethylation apparatus 1 may include various generators capable of generating high-power terahertz waves. For example, the generator may be a system that generates terahertz wave pulses using a crystal as in the embodiment, a system that generates continuous terahertz waves using a crystal or an air breakdown system. In addition, while a laser beam is used to generate a terahertz wave in the embodiment, the present invention is not limited thereto such that other means capable of generating a terahertz wave of a frequency band for inducing demethylation may be used.

The polarizer 600 may steer the terahertz wave radiated from the crystal 500 toward a predetermined direction using a reflective optical system. Here, the predetermined direction refers to an appropriate direction for irradiating the terahertz wave onto the sample 2.

The polarizer 600 shown in FIG. 1 includes two polarizing plate 601 and 602, two parabolic mirrors 603 and 604 and a reflector 604.

The intensity of the terahertz wave radiated from the crystal 500 may be adjusted by two polarizing plates 601 and 602. The terahertz wave passing through the polarizing plates 601 and 602 is steered and condensed by the parabolic mirror 603, the reflector 604 and the parabolic mirror 605 and then irradiated onto the sample 2.

The pyroelectric detector 700 measures the terahertz wave that has passed through the sample 2 with an optical sensor. Information about the measured terahertz wave is fed back to the laser beam generator 10, and the laser beam outputted by the laser beam generator 10 may be controlled according to the information.

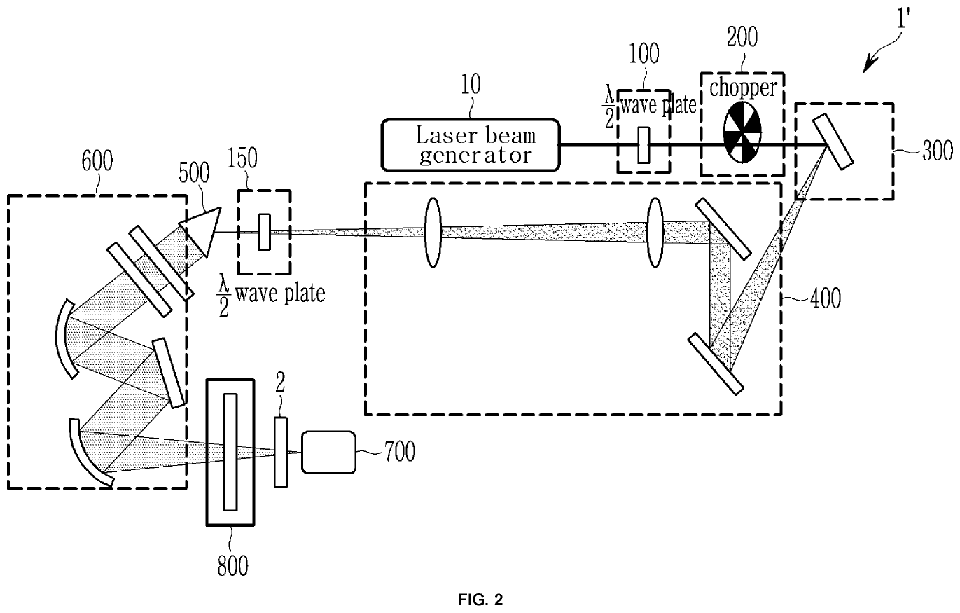

FIG. 2 illustrates a demethylation apparatus according to another embodiment of the present invention.

When compared with the embodiment shown in FIG. 1, the demethylation apparatus 1′ according to another embodiment further includes a filter 800. The filter 800 is located between the optical path between the polarizer 600 and the sample 3 and may selectively pass a terahertz wave of a predetermined frequency band outputted from the polarizer 600. The terahertz wave of the predetermined frequency band may cause more demethylation reaction than those of other frequency bands. According to an embodiment, the predetermined frequency band may be about 1.6 THz (1.5 to 1.7 THz), but is not limited thereto. For example, the predetermined frequency band may be a terahertz frequency range about methylation resonance frequency that generates the demethylation reaction as well as about 1.6 THz (1.5 to 1.7 THz).

According to the embodiments described with reference to FIGS. 1 and 2, the demethylation reaction occurs in the methylated sample 2 by the irradiated high-power terahertz wave. A terahertz wave measuring device for detecting the demethylation degree reaction is described below.

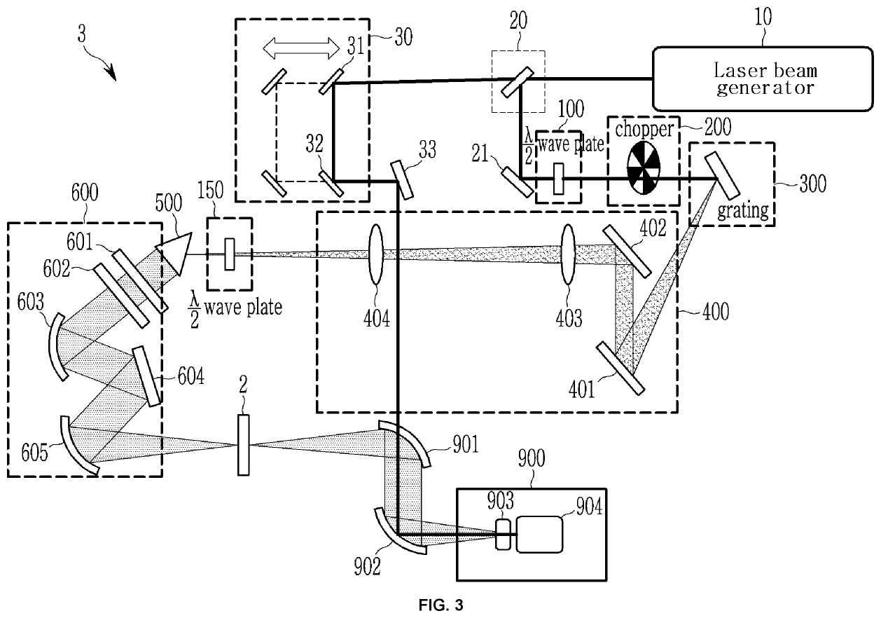

FIG. 3 illustrates a demethylation apparatus including a terahertz wave measuring device according to yet another embodiment of the present invention.

Like reference numerals indicates like parts described in FIG. 1.

As shown in FIG. 3, the demethylation apparatus 3 including a terahertz wave measuring device includes a laser beam generator 10, beam splitter 20, delay element 30, half wavelength plates 100 and 150, optical chopper 200, grating 300, a optical condenser 400, a crystal 500, a polarizer 600 and a pulse detector 900.

The description of the parts the same as those described with reference to FIG. 1 will be omitted.

The laser beam pulse from the laser beam generator 10 is split into two beams by the beam splitter 20. One of the two beams is a pump beam that excites the crystal 500 to generate a terahertz wave, and the other of the two beams is a probe beam that is irradiated to the pulse detector 900 to detect terahertz wave pulses. The terahertz wave pulse generated by the crystal 500 is incident on the sample, and then reflected from and/or transmitted through the sample. The terahertz wave pulse is modified by the reflection and/or transmission. The modified terahertz wave pulse is detected by the pulse detector 900.

The pump beam generated by the beam splitter 20 is steered by the reflector 21, and passes through the half wavelength plate 100, optical chopper 200, grating 300 and optical condenser 400 to be incident upon the crystal 500.

The delay element 30 is necessary for sampling the form of the terahertz wave pulse on a time axis, and generates the probe beam by delaying the laser beam outputted from the laser beam generator 10 and changing the path thereof. The probe beam may be used to scan and sample the terahertz wave pulses passed through the sample 2.

Since the length of the optical path is varied by the delay element 30, the time it takes for the probe beam to reach the pulse detector 900 is also varied. The embodiment may be applied to time-domain spectroscopy (TDS) for measuring the intensity of terahertz wave pulses incident on the pulse detector 900 while varying time.

The delay element 30 includes two reflectors 31 and 32, and the positions of the two reflectors 31 and 32 may be changed along the first axis (direction of the arrow shown in FIG. 3). As a result, the length of the optical path of the probe beam is varied, and the change when the terahertz wave pulse passes through the sample 2 on the time axis may be measured.

The parabolic mirrors 901 and 902 condense terahertz wave pulses that have passed through the sample 2 and the condensed terahertz wave pulses are incident on the pulse detector 900. The probe beam from the reflector 33 passes through the parabolic mirror 901, is reflected by the parabolic mirror 902, and is incident on the pulse detector 900.

The pulse detector 900 detects the terahertz wave pulse that has passed through the sample 2. The pulse detector 900 measures the intensity of the incident terahertz wave pulse according to the change over time. The pulse detector 900 scans and samples the terahertz wave pulse that has passed through the sample 2 using the probe beam to measure a change in the terahertz wave pulse. When the signal corresponding to the intensity of the terahertz wave pulse measured by the pulse detector 900 is subjected to Fourier transform, the amplitude and phase of the spectrum of the transmitted terahertz wave may be obtained.

The pulse detector 900 may sample the terahertz wave pulses using an electro-optic sampling method. The pulse detector 900 includes a nonlinear crystal (e.g., ZnTe) 903 and a balanced photodetector 904.

The terahertz wave pulses as well as the laser beam transmitted through the sample 2 are incident on the nonlinear crystal 903. When the laser beam, which is the probe beam, and the terahertz wave pulses are incident on the nonlinear crystal 903, a phenomenon in which the polarization of the laser beam may change according to the electric field of the terahertz wave pulse due to the Pockels effect.

The photo detector 904 may indirectly measure the terahertz wave pulses by detecting a change in polarization of the laser beam using a quarter wavelength plate, a beam splitting polarizer, and a balance photodiode.

The photodetector 904 may measure the change over time in the intensity of the terahertz wave pulses according to TDS, and detect the amplitude and phase of each frequency component by subjecting the measured intensity to Fourier transform.

A demethylation reaction is induced by irradiating the high-power terahertz wave on the sample using the demethylation apparatuses shown in FIGS. 1 and 2. The demethylation degree can be measured by the terahertz wave measuring device of the demethylation apparatus shown in FIG. 3.

When the terahertz wave is incident on methylated molecules, the resonance peak of the spectrum of the terahertz wave in a predetermined frequency band is determined according to the methylation degree of the methylated molecules. For example, as the methylation degree of DNA increases, the resonance peak gets higher around 1.6 THz (between 1.5 and 1.7 THz) in the frequency spectrum of the terahertz wave. Therefore, the demethylation degree of the methylated sample demethylated by the demethylation apparatus may be obtained through the terahertz wave measuring device.

Hereinafter, the effects of the demethylation apparatus will be described through experiment examples.

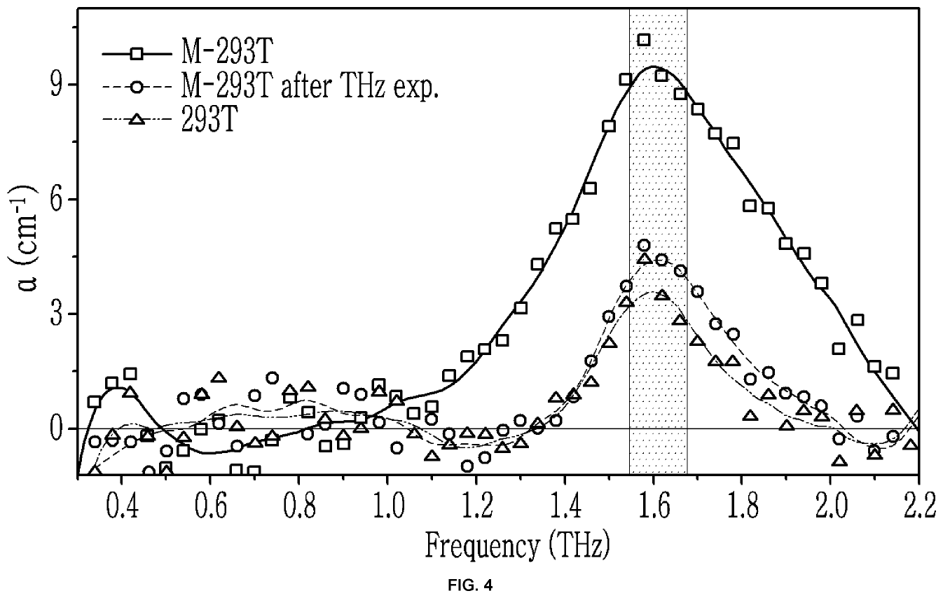

FIG. 4 is a graph showing the demethylation degree of DNA demethylated by high-power terahertz wave using the demethylation apparatus shown in FIG. 2.

In the graph shown in FIG. 4, the horizontal axis represents the frequency of the terahertz wave spectrum, and the vertical axis represents the terahertz wave absorption rate of the sample.

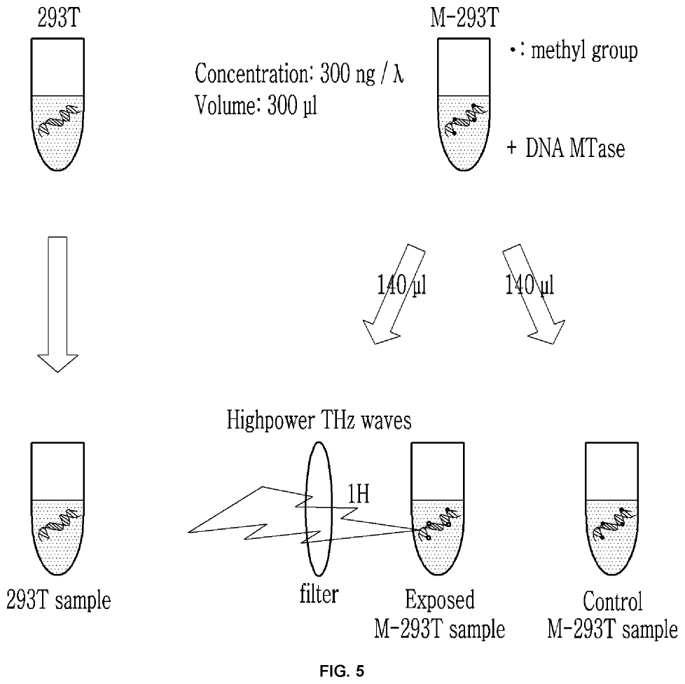

FIG. 5 illustrates an experiment for demonstrating the effect of demethylation by manufacturing three samples.

As shown in FIG. 5, HEK293T DNA (denoted as “293T”, human kidney cell line) and artificially methylated HEK293T DNA (denoted as “M-293T”, methylated 293T) were prepared. In this case, DNA is genomic DNA extracted from cells.

The M-293T is divided into two samples, and a high-power terahertz wave is irradiated onto one of the M-293T samples for 1 hour using the demethylation apparatus shown in FIG. 2. In this case, the filter used passes a terahertz wave in approximately 1.5 THz band.

As a result, as shown in FIG. 5, a non-methylated 293T sample (denoted as “293T sample”), an M-293T sample exposed to the high-power terahertz wave (denoted as “Exposed M-293T sample”) and an M-293T sample not exposed to the high-power terahertz wave (denoted as “Control M-293T sample”) were be obtained.

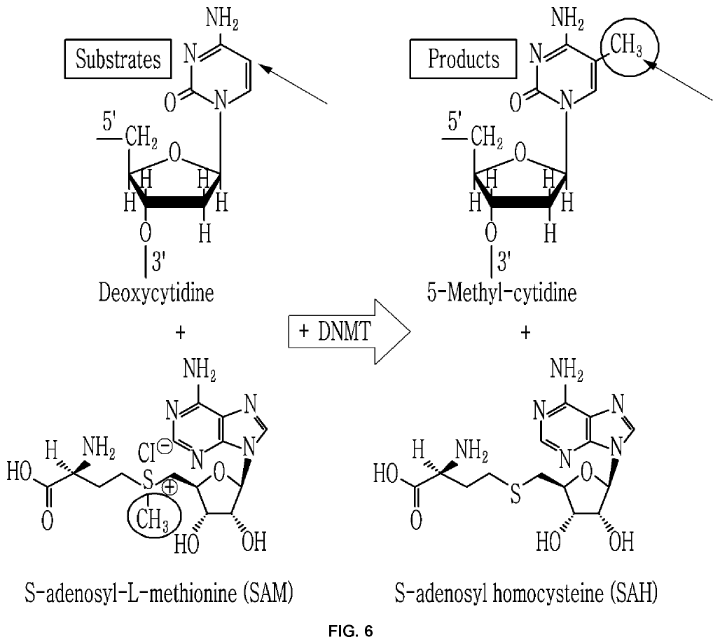

FIG. 6 illustrates a method of manufacturing an M-293T.

As shown in FIG. 6, according to the in-vitro method of manufacturing M-293T, a methyl group is bound to 293T using DNMT enzyme.

Deoxycytidine and S-adenosyl-L-methionine (SAM) were prepared as substrates, and DNMT (DNA methyltransferase; New England Biolab) was added to prepare 5-Methyl-cytidine with a methyl group bonded to the 5 position of the cytosine according to the manufacturer's manual (https://www.neb.com/products/m0230-human-dna-cytosine-5-methyltransferase-dnmt1#Protocols %20&%20Manuals). This methylation process of Deoxycytidine is represented by the reaction scheme shown in FIG. 6.

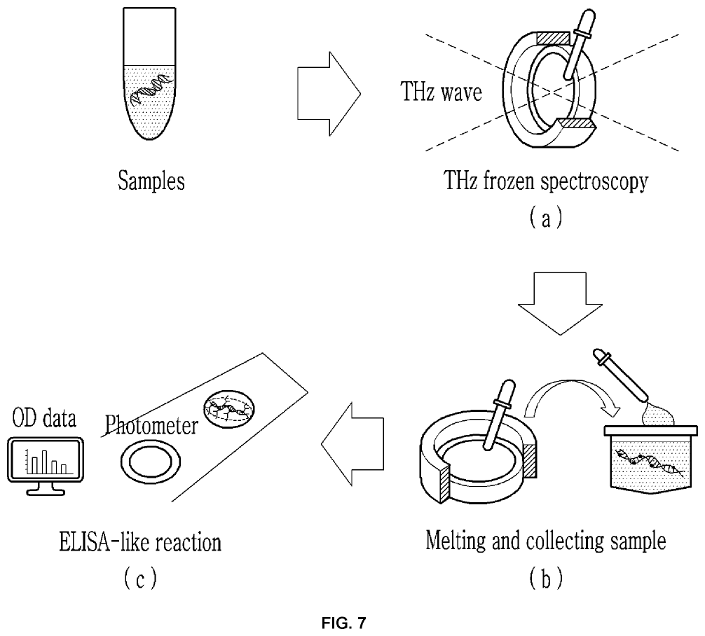

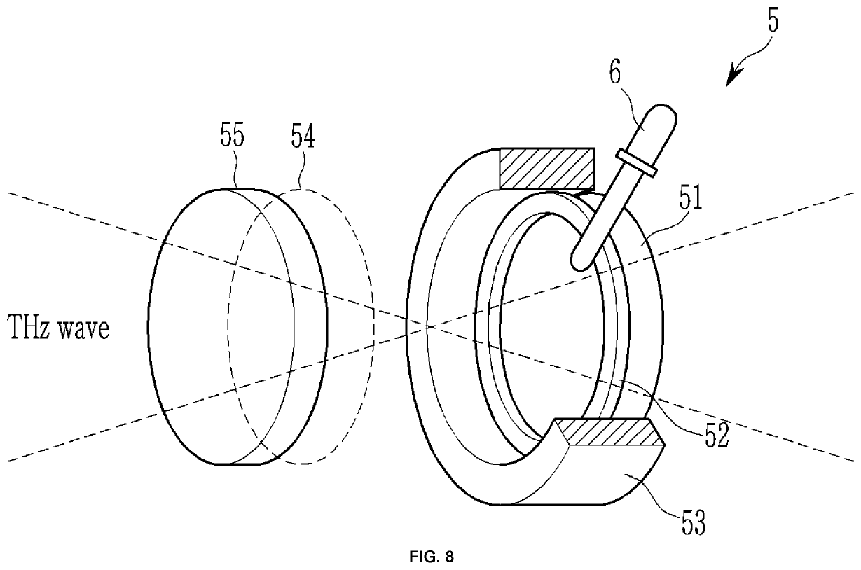

FIG. 7 illustrates a method of measuring demethylation results, and FIG. 8 illustrates a sample holder.

The intensity of the terahertz wave may be rapidly reduced by the moisture in the sample. In the experiment, a sample holder was used along with the terahertz wave measuring device of the demethylation apparatus. As shown in FIG. 8, a sample is injected into a sample holder 5 via the pipette 6, and the sample is frozen together with the sample holder 5 at minus 20 degrees.

For example, as shown in FIG. 8, the sample holder 5 includes a quartz window 51 having a hydrophobic surface 54, a copper spacer 52, a thermoelectric cooler 53 and a Teflon window 55.

The sample placed between the hydrophobic surface 54 of the quartz window 51 and the Teflon window 55 is cooled to 20 degrees below zero by the thermoelectric cooler 53.

As shown in (a) of FIG. 7, by using the terahertz wave measuring device of the demethylation apparatus shown in FIG. 3, a terahertz TDS was performed on the three samples having a thickness of 300 μm at −20° C., and the spectrum shown in FIG. 4 was obtained. In order to maintain the temperature constant during the terahertz TDS, the sample holder as shown in FIG. 8 was used.

According to the spectrum shown in FIG. 4 obtained by performing the terahertz TDS, the resonance peak for the methylation of the DNA is proportional to the methylation degree of the DNA. The resonance peak is located at approximately 1.6 THz (between 1.5 and 1.7 THz). According to the result of the experiment, the resonance peak of M-293T sample irradiated with high-power terahertz wave (using 1.5 THz filter) decreases to a level similar to that of 293T sample.

As shown in (b) of FIG. 7, the samples are melted and recovered, and as shown in (c) of FIG. 7, the result of the experiment is verified via biological measurement such as the Elisa-like reaction method using the recovered sample.

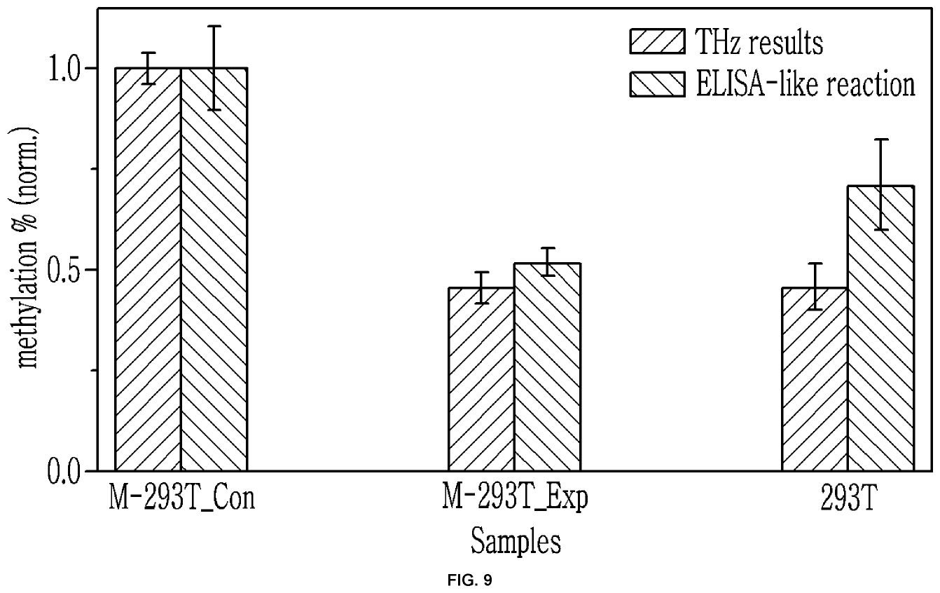

FIG. 9 is a graph showing the methylation degrees of the three samples verified by ELISA-like reaction.

The “THz result” bar graphs denoted as “THz result” represent the methylation degree of DNA of the three samples measured using the terahertz wave measuring device, and the bar graphs denoted as “ELISA-like reaction” represent the methylation degree of the DNA of the three samples measured by the Elisa-like reaction method (hereinafter, referred to as ELISA quantitative method).

As shown in FIG. 9, the methylation degrees of the DNA of the M-293T sample exposed to the terahertz wave (denoted as “M-293T_Exp”) measured using the terahertz wave measuring device and the ELISA quantitative method are almost the same. In addition, the methylation degree of the M-293T sample (denoted as “M-293T_Exp”) is very low compared to the DNA methylation degree of the M-293T sample not exposed to the terahertz wave (denoted as “M-293T_Con”), almost the same as the methylation degree of the non-methylated 293T sample (denoted as “293T”).

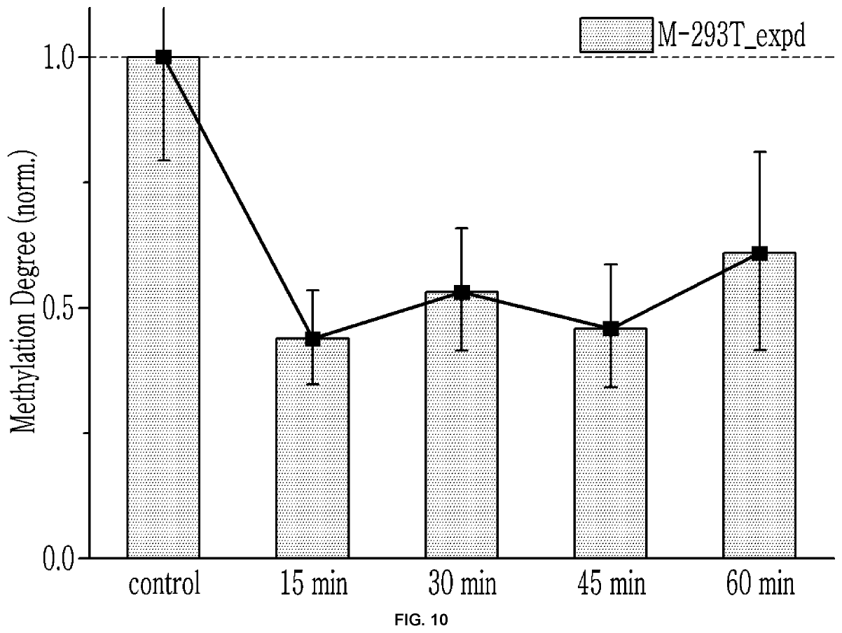

FIG. 10 is a graph showing the demethylation degree according to exposure time to high-power terahertz wave.

As shown in FIG. 10, M-293T sample was divided into five groups, that is, an M-293T sample (denoted as “control”) not exposed to high-power terahertz wave, an M-293T sample (denoted as “15 min”) exposed to high-power terahertz wave for 15 minutes, an M-293T sample (denoted as “30 min”) exposed to high-power terahertz wave for 30 minutes, an M-293T sample (denoted as “45 min”) exposed to high-power terahertz wave for 45 minutes and an M-293T sample (denoted as “60 min”) exposed to high-power terahertz wave for 60 minutes.

As shown in FIG. 10, when the methylation degree of the M-293T sample not exposed to-power terahertz wave is 1, about 50% demethylation occurs before the exposure time of 15 minutes elapses. After 15 minutes of exposure, the methylation degrees of the M-293T samples exposed to high-power terahertz waves for 30 minutes, 45 minutes and 60 minutes increase and decrease repeatedly, but remains within a certain range.

As shown in FIG. 10, about 15 minutes of exposure time to the high-power terahertz wave may be most appropriate. However, the present invention is not limited thereto, and the exposure time to the high-power terahertz wave may be appropriately selected according to the experiment conditions and target cells.

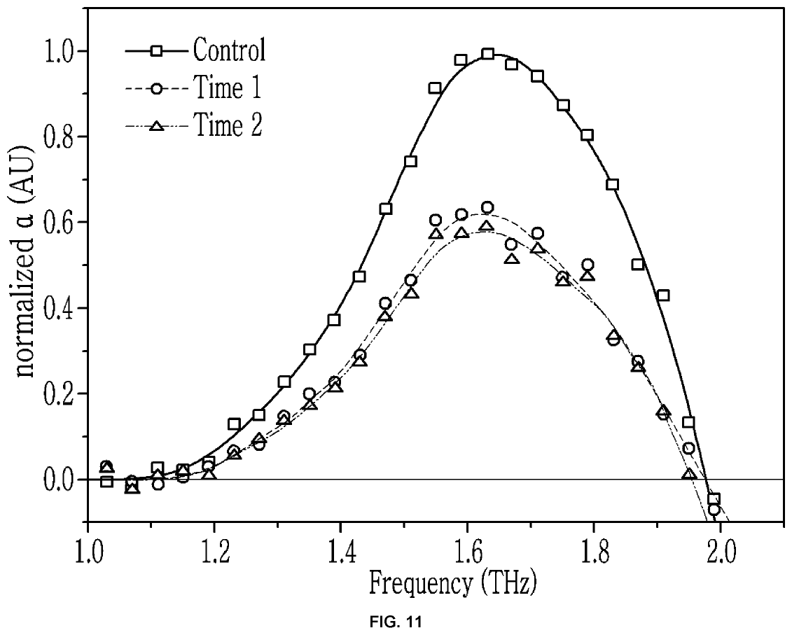

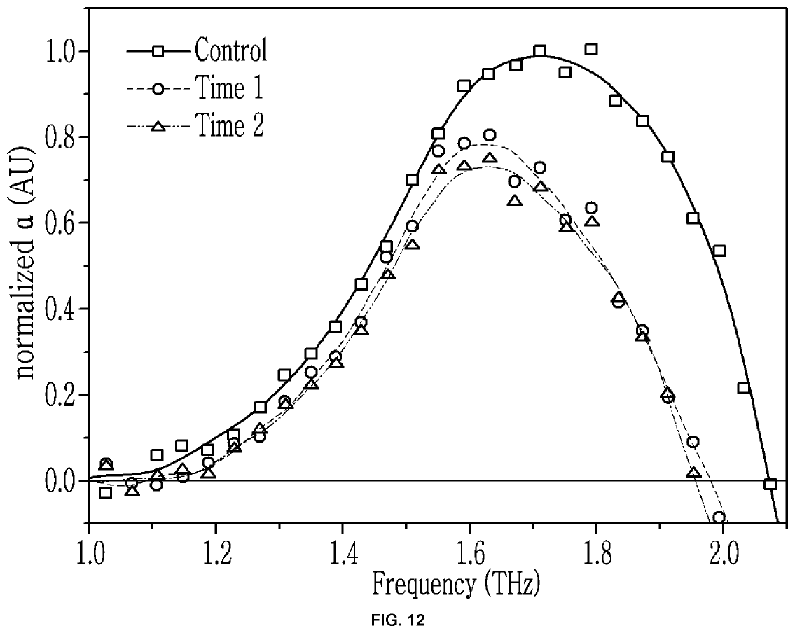

FIG. 11 is a graph showing the demethylation degree according to a high-power terahertz wave according to the embodiment shown in FIG. 1, and FIG. 12 is a graph showing the demethylation degree according to a high-power terahertz wave according to the embodiment shown in FIG. 2.

FIG. 11 is a graph showing the demethylation degrees of the three samples exposed to high-power terahertz wave generated by the demethylation apparatus shown in FIG. 1, and FIG. 12 is a graph showing the demethylation degrees of the three samples exposed to high-power terahertz wave generated by the demethylation apparatus shown in FIG. 2.

It is to be demonstrated that, with reference to FIGS. 11 and 12, the demethylation reaction using high-power terahertz wave is caused by a resonance phenomenon. In the case of the embodiment shown FIG. 2, side effects that may be caused by high-power terahertz waves of other frequency bands may be prevented in advance by excluding electromagnetic waves of other frequency bands by the filter.

In the experiment in which the graphs shown FIGS. 11 and 12 were derived, 5-methylcytidine (5-mC) methylated with cytidine in DNA was used. This is the part that changes directly in the methylation process of DNA. The experiment was the same as the experiment method described with reference to FIGS. 5 through 8, and, using a 5-mC aqueous solution (30 mg/300 μl), an experiment sample (denoted as “Time 1”) irradiated with high-power terahertz wave for a first time period, another experiment sample (denoted as “Time 2”) irradiated with high-power terahertz wave for a second time period different from the first time period and a reference sample (denoted as “Control”) not radiated with high-power terahertz wave were used.

The spectrum shown in FIG. 11 was obtained from: the experiment sample (Time 1) irradiated with high-power terahertz wave for the first time period using the demethylation apparatus (without filter) shown in FIG. 1; the experiment sample (Time 2) irradiated with high-power terahertz wave for the second time period using the demethylation apparatus (without filter) shown in FIG. 1; and the reference sample (Control) not irradiated with any terahertz wave. The resonance peak is located about 1.6 THz, which is the same as that of the aqueous DNA solution. In FIG. 11, the heights of the resonance peaks represent the concentration of 5-mC that maintains the original molecular structure.

As shown in FIG. 11, the experiment sample (Time 1) and the experiment sample (Time 2) both showed about the same level of demethylation such that the resonance peaks thereof are much lower than that of the reference sample (Control). That is, it is seen that the irradiation of high-power terahertz waves for a predetermined time period or longer may induce the demethylation reaction.

The spectrum shown in FIG. 12 was obtained from: the experiment sample (Time 1) irradiated with high-power terahertz wave for the first time period using the demethylation apparatus (with filter) shown in FIG. 2; the experiment sample (Time 2) irradiated with high-power terahertz wave for the second time period using the demethylation apparatus (with filter) shown in FIG. 2; and the reference sample (Control) not irradiated with any terahertz wave. As shown, the resonance peak is located around 1.6 THz which is the same as that of the aqueous DNA solution. In FIG. 12, the heights of the resonance peaks represent the concentration of 5-mC that maintains the original molecular structure.

As shown in FIG. 12, the experiment sample (Time 1) and the experiment sample (Time 2) both showed about the same level of demethylation such that the resonance peaks thereof are much lower than that of the reference sample (Control) similar to the graph shown in FIG. 11. That is, it is seen that the irradiation of high-power terahertz waves for a predetermined time period or longer may induce the demethylation reaction.

As described above, the high-power terahertz wave when radiated onto a sample induces the demethylation reaction. Considering the decrease of the resonance peak even when the filter is applied, it can be seen that the high-power terahertz wave of the predetermined frequency band (approximately 1.6 THz) is deeply involved in inducing demethylation.

The reason the demethylation degrees of the samples irradiated with terahertz wave using filter as shown in FIG. 12 is lower than those of the samples irradiated with terahertz wave without using filter as shown in FIG. 11 is that the power of the terahertz wave in 1.6 THz band is reduced by about 25% with filter.

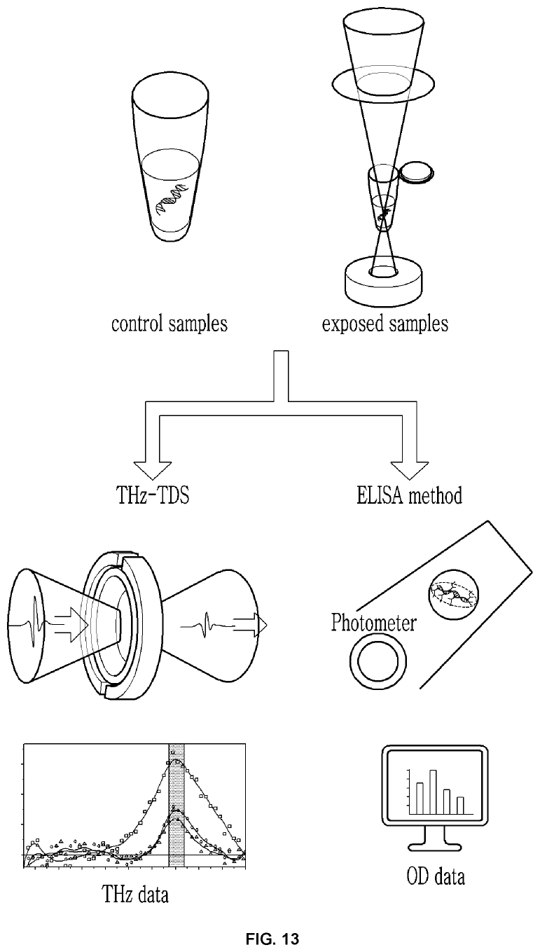

FIG. 13 schematically illustrates a process of demethylation experiment.

FIG. 13 is a schematic diagram of a process of measuring methylation degree using a terahertz wave measuring device and an ELISA method for each of a methylated cell line sample (control samples) and a cell line sample exposed to a high-power terahertz wave (exposed samples). It is a drawing shown as.

14 to 18 are diagrams showing a graph showing a resonance frequency of a blood cancer sample and a resonance frequency of a demethylated sample, and a graph showing a comparison and verification of the methylation degree using a terahertz wave measuring device and ELISA quantification method.

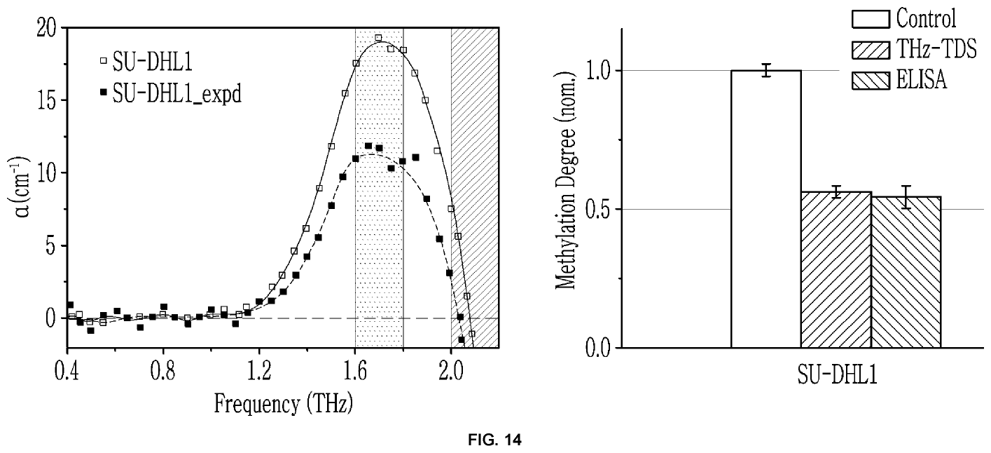

FIGS. 14 through 18 illustrate graphs depicting resonance frequency of a blood cancer sample and resonance frequency of a demethylated blood cancer sample, and bar graphs depicting methylation degrees of the samples measured by the terahertz wave measuring apparatus and verified by ELISA quantitative method.

As shown in FIGS. 14 through 18, the methylation degrees of the cell line samples (denoted as “SU-DHL1_expd”, “SU-DHL9_expd”, “OCI-LY1_expd”, “Raji_expd”, “CCRF-CEM_expd”, “Jurkat_expd” and “HL-60_expd”) exposed to the high-power terahertz wave are lower than those of the cell line samples (denoted as “SU-DHL1”, “SU-DHL9”, “OCI-LY1”, “Raji”, “CCRF-CEM”, “Jurkat” and “HL-60”) not exposed to any terahertz wave.

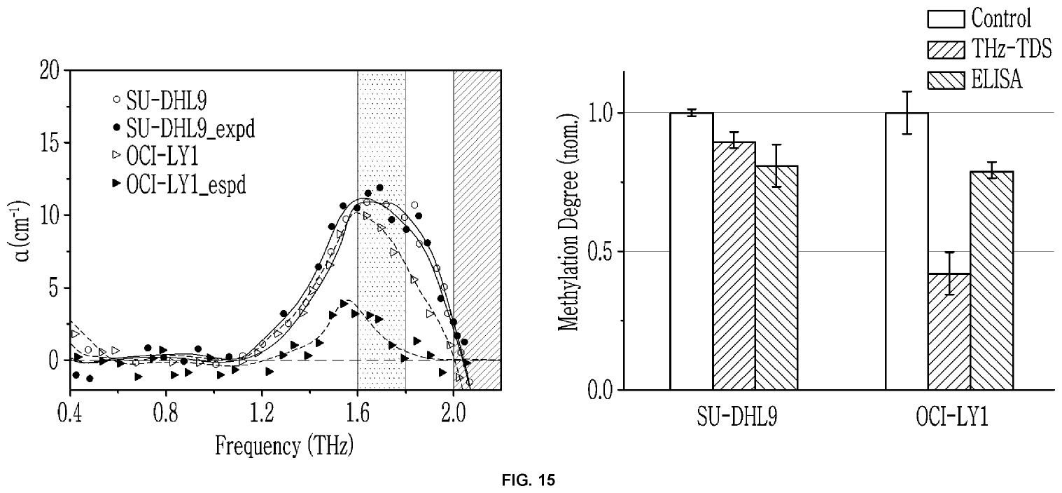

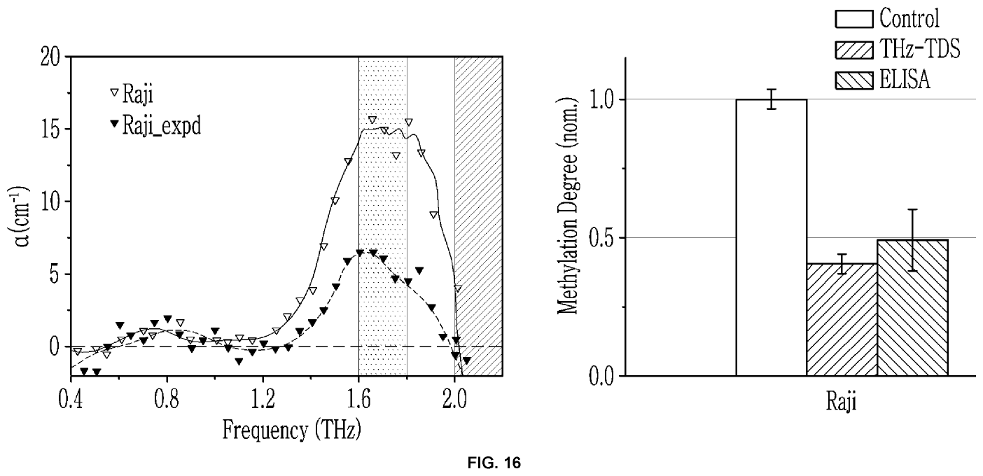

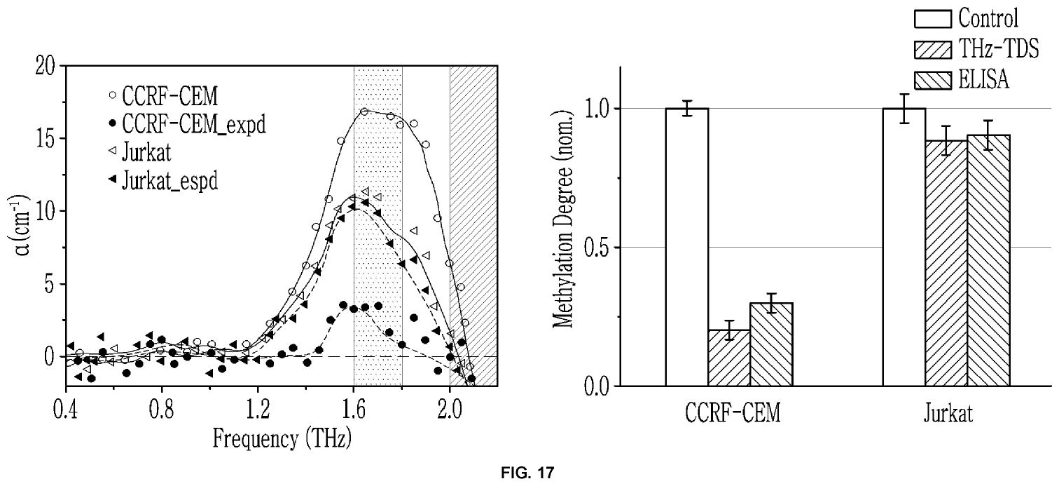

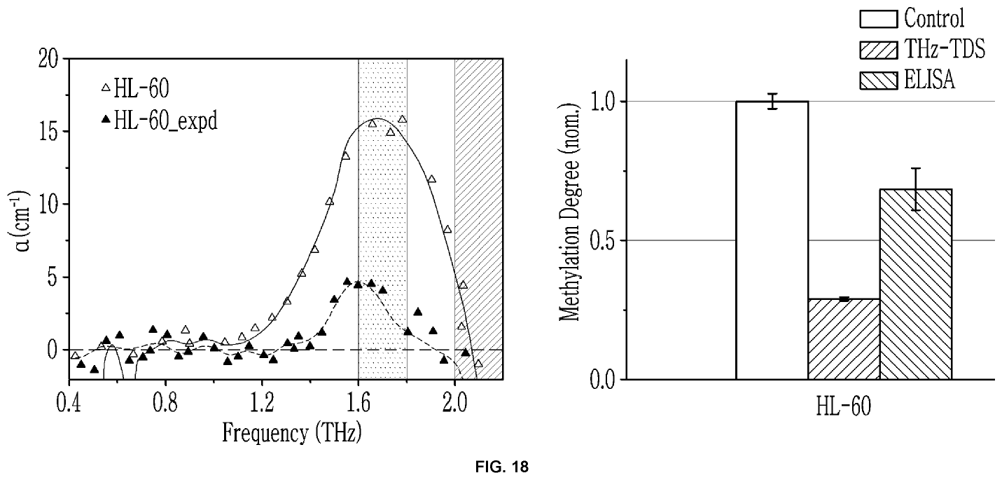

“SU-DHL1” in FIG. 14 refers to a T-cell lymphoma, “SU-DHL9” and “OCI-LY1” in FIG. 15 refer to B-cell lymphomas “Raji” in FIG. 16 refers to Burkitt's lymphoma, CCRF-CEM″ and “Jurkat” in FIG. 17 refers to T-cell acute lymphocytic leukemia, and “HL-60” in FIG. 18 refers to acute myeloid leukemia.

In the bar graphs of FIGS. 14 through 18, “Control” represents the methylation degrees of the cell line samples (SU-DHL1, SU-DHL9, OCI-LY1, Raji, CCRF-CEM, Jurkat and HL-60) not exposed to any terahertz wave, “THz-TDS” represents the methylation degrees of the cell line samples (SU-DHL1_expd, SU-DHL9_expd, OCI-LY1_expd, Raji_expd, CCRF-CEM_expd, Jurkat_expd and HL-60_expd) exposed to high-power terahertz wave measured using the terahertz wave measuring device, and “ELISA” represents the methylation degrees of the cell line samples (SU-DHL1_expd, SU-DHL9_expd, OCI-LY1_expd, Raji_expd, CCRF-CEM_expd, Jurkat_expd and HL-60_expd) exposed to high-power terahertz wave measured by ELISA quantitative method

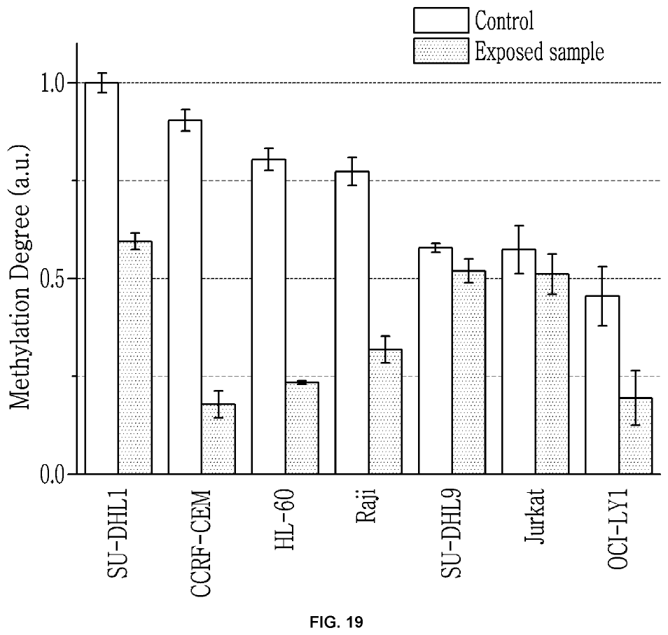

FIG. 19 is a graph showing that demethylation actually occurs in leukemia cell line when irradiated with high-power terahertz wave.

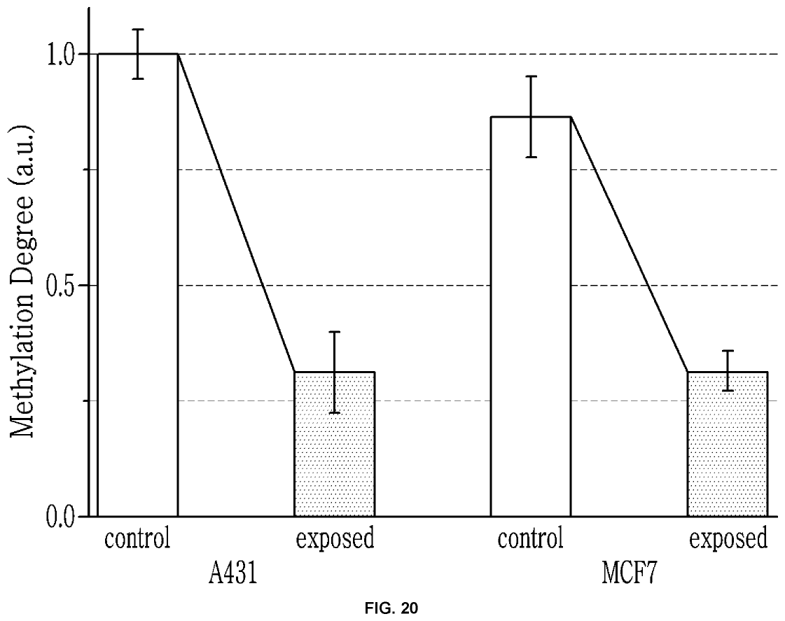

FIG. 20 is a graph showing that demethylation actually occurs in skin cancer cell line and breast cancer cell line when irradiated with high-power terahertz wave.

In FIG. 20, A431 denotes an exemplary skin cancer cell line, and MCF7 denotes an exemplary breast cancer cell line.

As shown in FIGS. 19 and 20, the methylation degrees of the cell line samples exposed to high-power terahertz wave (denoted as “Exposed sample”) are lower than those of the cell line sample (denoted as “Control”) not exposed to the terahertz wave.

The above-described demethylation apparatus and the demethylation method may be applied to a cancer therapy method and apparatus.

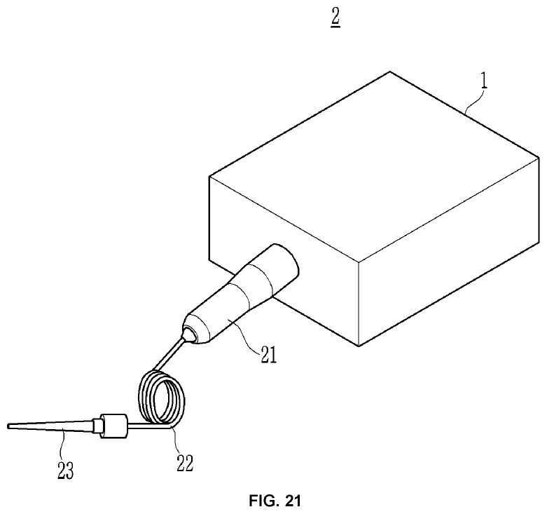

FIG. 21 illustrates a cancer therapy apparatus according to an embodiment of the present invention.

As shown in FIG. 21, the cancer therapy apparatus 2 includes the demethylation apparatus 1, a terahertz wave lens 21, a terahertz wave waveguide 22 and a terahertz wave output tube 23.

The demethylation apparatus 1′ may be employed in cancer therapy apparatus 2 in place of demethylation apparatus 1.

The terahertz wave lens 21 condenses the high-power terahertz wave outputted from the demethylation apparatus 1.

The high-power terahertz wave condensed by the terahertz wave lens 21 travels along the waveguide 22. a high-power terahertz wave traveled along the waveguide 22 is irradiated onto the affected area where cancer cells are present via the terahertz wave output tube 23. The waveguide 22 may be extended to a predetermined length such that the terahertz wave output tube 23 may be positioned on the affected area. The user may hold and move the terahertz wave output tube 23 for treatment toward the affected area. The terahertz wave output tube 23 may be have a shape suitable for enabling such manipulation.

The embodiment shown in FIG. 21 is an exemplary cancer therapy apparatus, and the present invention is not limited thereto.

Although the embodiment of the present invention has been described in detail above, the scope of the present invention is not limited thereto, and various modifications and improvements by those skilled in the art using the basic concept of the present invention defined in the following claims are also in the scope of the present invention.

What is claimed is:

1. A demethylation method, comprising:
(a) irradiating a terahertz wave of a first frequency band including a methylation resonance frequency onto a methylated molecule; and
(b) demethylating the methylated molecule by the terahertz wave.

2. The demethylation method of claim 1, further comprising:
(c) generating the terahertz wave using a laser beam; and
(d) condensing and steering the terahertz wave by a polarizer toward the methylated molecule to be irradiated in (a).

3. The demethylation method of claim 2, further comprising:
(e) filtering the terahertz wave condensed and steered in (d) to pass the terahertz wave of the first frequency band.

4. The demethylation method of claim 1, wherein the first frequency band comprises a frequency of 1.6 THz.

5. A cancer therapy method comprising a demethylation method of claim 1.

6. A cancer therapy apparatus capable of performing a demethylation method of claim 1.

7. A demethylation apparatus irradiating a terahertz wave of a first frequency band including a methylation resonance frequency onto a methylated molecule to demethylate the methylated molecule by the terahertz wave.

8. The demethylation apparatus of claim 7, comprising:
a laser beam generator generating a laser beam;
a crystal excited by the laser beam to generate the a terahertz wave;
a polarizer condensing and steering the terahertz wave generated by the crystal toward the methylated molecule to be irradiated onto the methylated molecule.

9. The demethylation apparatus of claim 8, further comprising: a filter passing the terahertz wave of the first frequency band.

10. The demethylation apparatus of claim 9, wherein the filter is positioned in an optical path between the polarizer and the methylated molecule

11. The demethylation apparatus of claim 9, further comprising:
a first half wavelength plate changing a polarization direction of the laser beam generated by the laser beam generator by 90 degrees; and
a grating 300 tilting the laser beam having the polarization direction thereof changed by 90 degrees toward the crystal to be incident thereon.

12. The demethylation apparatus of claim 11, further comprising: an optical condenser disposed in an optical path between the grating and the crystal and condensing the laser beam from the grating to be incident on the crystal.

13. The demethylation apparatus of claim 12, further comprising: a second half wavelength plate disposed in an optical path between the optical condenser and the crystal and changing a polarization direction of the laser beam condensed by the optical condenser to a vertical polarization.

14. The demethylation apparatus of claim 7, wherein the terahertz wave comprises terahertz wave pulses or a continuous terahertz wave.

15. The demethylation apparatus of claim 7, wherein the first frequency band comprises a frequency of 1.6 THz.

16. A cancer therapy apparatus comprising a demethylation apparatus of claim 7.
